(19) United States
(12) Patent Application Publication
(10) Pub. No.: US 2026/0347833 A1
(43) Pub. Date: Nov. 5, 2026

(54) METHODS OF PRODUCING LARGE-SCALE PLASMID LIBRARIES

(71) Applicants: The General Hospital Corporation, Boston, MA (US); THE BROAD INSTITUTE, INC., Cambridge, MA (US)

(72) Inventors: Marcela V. Maus, Lexington, MA (US); Robert Manguso, Boston, MA (US); Nelson Knudsen, Boston, MA (US)

(73) Assignees: The General Hospital Corporation, Boston, MA (US); The Broad Institute, Inc., Cambridge, MA (US)

(21) Appl. No.: 19/486,896

(22) PCT Filed: May 23, 2024
(86) PCT No.: PCT/US2024/030764

§ 371 Date: Nov. 21, 2025

Related U.S. Application Data
(60) Provisional application No. 63/503,921, filed on May 23, 2023.

(51) Int. Cl. C12N 15/10

(52) U.S. Cl. CPC C12N 15/1093

(57) ABSTRACT

Provided herein are methods of producing plasmid libraries according to Current Good Manufacturing Practice (cGMP) guidelines.

### RELATED APPLICATIONS

This application claims the benefit under 35 U.S.C. § 119 (e) to U.S. Provisional Application No. 63/503,921, filed May 23, 2023, entitled “Methods of Producing Large-Scale Plasmid Libraries”, the entire contents of which are incorporated herein by reference.

### BACKGROUND

Plasmid production according to current Good Manufacturing Practice (cGMP) involves a series of stringent processes to ensure the quality, safety, and consistency of the final product. This typically includes the selection of a high-quality bacterial strain for plasmid propagation, controlled fermentation under optimal conditions to maximize plasmid yield, and purification using validated methods to remove impurities and endotoxins.

### SUMMARY

To date, protocols that adhere to Current Good Manufacturing Practice (cGMP) guidelines for plasmid production have been limited to producing homogeneous plasmids (e.g. a plasmid pool wherein each plasmid comprises the same genetic material). Thus, large-scale library-based assays cannot be performed while conforming to cGMP, which is required for clinical products.

Accordingly, provided herein in some aspects is a method of producing a Current Good Manufacturing Practice (cGMP) plasmid library comprising a plurality of different plasmids, the method comprising transforming the plurality of different plasmids into a plurality of competent E. coli, wherein the plasmids comprise a nucleic acid encoding an antibiotic resistance gene; contacting the transformed competent E. coli with a first culture and achieving a minimum number of colonies per each of the different plasmids, wherein the first culture comprises an antibiotic corresponding to the antibiotic resistance gene; extracting the colonies from the first culture; growing the extracted colonies in a second culture according to cGMP guidelines, wherein the second culture comprises an antibiotic that corresponds to the antibiotic resistance gene, wherein the antibiotic is present at a concentration at least twice the standard concentration of the antibiotic; and extracting the plasmids from the second culture according to cGMP guidelines to produce a cGMP plasmid library.

In some embodiments, the minimum number of colonies is about 100 colonies per each of the different plasmids. In some embodiments, the minimum number of colonies is about 1000 colonies per each of the different plasmids.

In some embodiments, the plasmid library comprises a plasmid that encodes a RNA or a protein. In some embodiments, the plasmid library comprises a plasmid that encodes a therapeutic RNA or a therapeutic protein.

In some embodiments, the plasmid library comprises at least about 5 different plasmids. In some embodiments, the plasmid library comprises at least about 50 different plasmids. In some embodiments, the plasmid library comprises at least about 100 different plasmids. In some embodiments, the plasmid library comprises at least about 250 different plasmids. In some embodiments, the plasmid library comprises at least about 500 different plasmids. In some embodiments, the plasmid library comprises at least about 1000 different plasmids.

In some embodiments, the competent E. coli comprise electrocompetent E. coli. In some embodiments, the competent E. coli comprise chemically-competent E. coli.

In some embodiments, the antibiotic is any one of ampicillin, carbenicillin, kanamycin, specinomycin, streptomycin, bleomycin, erythromycin, chloramphenicol, tetracycline, zeocin, D-cycloserine, gentamicin, G418, nalidixic acid, rifampicin, trimethoprim. In some embodiments, the antibiotic is a beta-lactam antibiotic. In some embodiments, the beta-lactam antibiotic is a penicillin antibiotic. In some embodiments, the penicillin antibiotic is a carboxypenicillin antibiotic. In some embodiments, the carboxypenicillin antibiotic is carbenicillin. In some embodiments, the carbenicillin concentration is at least about 150 μg/mL. In some embodiments, the carbenicillin concentration is at least about 200 μg/mL.

In some embodiments, the antibiotic and corresponding concentration is ampicillin: at least about 200 μg/mL; carbenicillin: at least about 200 μg/mL; kanamycin: at least about 200 μg/mL; spectinomycin: at least about 100 μg/mL; streptomycin: at least about 200 μg/mL; bleomycin: at least about 200 μg/mL; erythromycin: at least about 200 μg/mL; chloramphenicol: at least about 50 μg/mL; tetracycline: at least about 20 μg/mL; zeocin: at least about 200 μg/mL; D-cycloserine: at least about 400 μg/mL; gentamicin: at least about 30 μg/mL; G418: at least about 800 g/mL; nalidixic acid: at least about 60 μg/mL; rifampicin: at least 100 about μg/mL; or trimethoprim: at least about 400 μg/mL.

In some aspects, the disclosure provides a method of producing a Current Good Manufacturing Practice (cGMP) plasmid library comprising a plurality of different plasmids, the method comprising transforming the plurality of different plasmids into a plurality of electrocompetent E. coli, wherein the plasmids comprise a nucleic acid encoding a carbenicillin resistance gene; contacting the transformed competent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises at least about 200 μg/mL of carbenicillin; and extracting plasmids from the second culture according to cGMP guidelines to produce a cGMP plasmid library.

In some aspects, the disclosure provides a method of producing a Current Good Manufacturing Practice (cGMP) plasmid library comprising a plurality of different plasmids, the method comprising transforming the plurality of different plasmids into a plurality of electrocompetent E. coli, wherein the plasmids comprise a nucleic acid encoding a carbenicillin resistance gene; contacting the transformed electrocompetent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises about 200-400 μg/mL of carbenicillin; and extracting plasmids from the second culture according to cGMP guidelines to produce a cGMP plasmid library.

In some aspects, the disclosure provides a method of producing a Current Good Manufacturing Practice (cGMP) plasmid library comprising a plurality of different plasmids, the method comprising transforming the plurality of different plasmids into a plurality of electrocompetent E. coli, wherein the plasmids comprise a nucleic acid encoding a carbenicillin resistance gene; contacting the transformed competent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises about 200 μg/mL of carbenicillin; and extracting plasmids from the second culture according to cGMP guidelines to produce a cGMP plasmid library.

In some aspects, the disclosure provides a method of producing a Current Good Manufacturing Practice (cGMP) plasmid library comprising a plurality of different plasmids, the method comprising transforming the plurality of different plasmids into a plurality of electrocompetent E. coli, wherein the plasmids comprise a nucleic acid encoding a carbenicillin resistance gene; contacting the transformed competent E. coli with an agar plate and achieving at least about 1000 colonies per each of the different plasmids, wherein the agar plate comprises carbenicillin; extracting the colonies from the agar plate; growing the extracted colonies in a liquid culture according to cGMP guidelines, wherein the liquid culture comprises about 200 μg/mL of carbenicillin; and extracting plasmids from liquid culture according to cGMP guidelines to produce a cGMP plasmid library.

In some embodiments, the minimum number of colonies is about 100 colonies per each of the different plasmids. In some embodiments, the minimum number of colonies is about 1000 colonies per each of the different plasmids.

### BRIEF DESCRIPTION OF THE DRAWINGS

FIG. 1 relates to growth curves of bacteria transformed with library plasmids.

FIG. 2 shows gRNA plasmid library yield is poor using standard protocols that adhere to current Good Manufacturing Practice (cGMP) guidelines.

FIG. 3 relates to gRNA plasmid library yield after varying concentrations of antibiotic.

FIGS. 4A-4C relate to the representation of single guide RNA (sgRNA) distribution in gRNA library plasmid preps grown at varying concentrations of carbenicillin from glycerol stocks compared to the original plasmid library. FIG. 4A shows the distribution of gRNA frequency, and FIGS. 4B-4C show representation of original DNA (x-axis) compared to DNA from varying preps (y-axis) in straight from glycerol or grown in 100 μg/mL of antibiotic (FIG. 4B), 200 μg/mL of antibiotic, or 400 μg/mL of antibiotic (FIG. 4C).

FIG. 5 shows improved cGMP gRNA plasmid library yield accomplished using methods developed herein. The gel shows cGMP gRNA plasmid library after treatment with restriction enzymes ScaI and EcoRI.

FIG. 6 are histograms of sgRNA distributions from non-cGMP standard gRNA plasmid library preparation (H01-H04) or cGMP large scale Contract Research Organization (CRO) gRNA plasmid library (H05-H08).

FIG. 7 are histograms of sgRNA distributions of primary human T cells transduced with virus made from non-cGMP standard gRNA plasmid library preparation (GPP) or large scale CRO cGMP gRNA plasmid library preparation.

### DETAILED DESCRIPTION

Regulatory agencies, such as the Food & Drug Administration (FDA) in the United States, have developed guidelines known as Current Good Manufacturing Practice (cGMP) to ensure the quality of products for human consumption or use in humans. Such guidelines apply to facilities and institutions that produce these products and are stringent in order to protect consumers. To date, protocols that adhere to cGMP guidelines only enable the production of homogeneous plasmids (e.g, wherein each plasmid encodes the same sequence) at a time, with no methods for producing plasmid libraries (comprising two or more different plasmids, e.g., two or more plasmids encoding different sequences) according to cGMP guidelines, such as transduction of immune effector cells for therapeutic purposes in humans. Provided herein are methodologies to produce libraries of plasmids comprising a plurality of different plasmids, allowing for large-scale preparation for library-based approaches that conform to cGMP guidelines and therefore produce plasmid libraries for use in clinical applications.

Current Good Manufacturing Practice (cGMP)

Current Good Manufacturing Practice (cGMP) is a set of guidelines enforced by regulatory agencies (e.g. the Food & Drug Administration (FDA) in the United States) to ensure the quality, safety, and efficacy of a variety of products, including clinical products. These guidelines cover various aspects of production, such as personnel, facilities, equipment, documentation, and processes, and adherence to the guidelines assures the identity, quality, purity, and potency of clinical products. The FDA inspects manufacturing facilities to confirm that such facilities (such as contract research organizations, CROs) are fully compliant with cGMP requirements.

cGMP industry guidance for the use of microbial vectors for gene therapy ensures that material (e.g. DNA) that will be used in clinical products adhere to FDA regulations. In some embodiments, gene therapy products refer to products that exert their effect via altering genetic material or expression of genetic material in a cell to influence the cell phenotype. The term “gene” may refer to a nucleic acid sequence which is transcribed (DNA) to RNA in vitro or in vivo when operably linked to appropriate regulatory sequences. cGMP guidelines provided by the FDA for the use of microbial vectors (e.g. bacteria such as E. coli) for gene therapy production include specific culturing procedures, cell growth conditions, harvest conditions, purification steps, and process timing and intermediate storage.

Parameters measured in the purified final product to determine conformity to cGMP guidelines include, for example:
(i) sterile filtration of the samples and sample appearance, e.g. colorless, clear, and lacking visible particulates;
(ii) A260: A280 ratio, which should fall within a range of about 1.7-2.0 to confirm the purity of DNA in the sample and a lack of RNA;
(iii) presence of endotoxin (pathogenic components of Gram-negative bacteria), which should be less than 30 EU/mg and less than 5 EU/kg/dose for any parentally administered plasmid-derived product;
(iv) plasmid size (compared to starting plasmid to be purified);
(v) DNA homogeneity, as determined by a minimum percentage of supercoiled DNA (at least 75% supercoiled DNA);
(vi) residual RNA, residual single-stranded DNA, residual linear DNA, and residual chromosomal DNA, which should not be detected when testing 1 μg of a final plasmid preparation;
(vii) restriction enzyme fragment sizes for DNA comprising Inverted Terminal Repeats, to confirm the DNA identity;
(viii) protein concentration, as purified samples should not have detectable residual host cellular protein;
(ix) pH of final product, which should fall in the range of 7.5-8.5 if formulated in TE buffer (10 mM Tris-HCl, 1 mM EDTA); and
(x) bioburden, or the unexpected growth of other microbial species after a specified amount of time (e.g., five days), which should not be present.

Adherence to the above exemplary standards for cGMP guidelines ensure suitability of plasmids for use in humans. Examples of standards for plasmid preparation according to cGMP guidelines are as described herein and additionally readily accessible and known in the art. See, for example, U.S. Dept. of Health & Human Services, Food & Drug Administration, Center for Biologics Evaluation and Research. Recommendations for Microbial Vectors used for Gene Therapy, 2016 (https://www.fda.gov/files/vaccines, % 20blood %20&%20biologics/published/Recommendations-for-Microbial-Vectors-Used-for-Gene-Therapy--Guidance-for-Industry.pdf), which also includes non-binding recommendations and suggested practice for plasmid production, the entirety of which is incorporated herein by reference.

Current protocols that adhere to cGMP guidelines are drawn to producing only one gene therapy product (e.g. one guide RNA to target a gene in a cell for use in humans) at a time. Provided herein are methods for producing a cGMP plasmid library comprising a plurality of different (e.g., encoding different sequences, such as different guide RNAs) plasmids. Plasmid libraries produced according to methods provided by the present disclosure allow for the production of cGMP libraries, e.g., for use in large-scale transduction or transformation of immune effector cells (such as T cells) that suitable for use in humans. Such methods represent a major contribution to the field as they may be utilized to generate any cGMP plasmid library for products suitable for use in humans.

In some aspects, this disclosure provides a method of producing a cGMP plasmid library comprising a plurality of different plasmids, the method comprises: transforming the plurality of different plasmids into a plurality of competent E. coli, wherein the plasmids comprise a nucleic acid encoding an antibiotic resistance gene; contacting the transformed competent E. coli with a first culture and achieving a minimum number of colonies per each of the different plasmids, wherein the first culture comprises an antibiotic corresponding to the antibiotic resistance gene; extracting the colonies from the first culture; growing the extracted colonies in a second culture according to cGMP guidelines, wherein the second culture comprises an antibiotic that corresponds to the antibiotic resistance gene, wherein the antibiotic is present at a concentration at least twice the standard concentration of the antibiotic; and extracting plasmids from the second culture according to cGMP guidelines to produce a cGMP plasmid library.

As used herein, “contacting” refers to combining a cell or cells with a culture medium, for example an agar plate or a liquid culture (e.g., a broth). “Extracting” colonies relates to isolating colonies from a culture medium, for example by scraping colonies off an agar plate or centrifuging a liquid culture and to separate the colonies from the liquid medium for isolation. “Extracting” plasmids refers to isolating plasmids from a culture (e.g., a culture of E. coli).

A “first culture” refers to a first culture medium that will facilitate the growth of the E. coli, e.g., an agar plate or a liquid culture. A “second culture” refers to a second culture medium that will facilitate the growth of E. coli, e.g., an agar plate or a liquid culture. In some embodiments, a first culture comprises an agar plate and a second culture comprises an agar plate. In some embodiments, a first culture comprises an agar plate and a second culture comprises a liquid culture. In some embodiments, a first culture comprises a liquid culture and a second culture comprises a liquid culture. In some embodiments, a first culture comprises a liquid culture and a second culture comprises an agar plate.

In some aspects, a method of producing a cGMP plasmid library comprising a plurality of different plasmids comprises: transforming the plurality of different plasmids into a plurality of electrocompetent E. coli, wherein the plasmids comprise a nucleic acid encoding a carbenicillin resistance gene; contacting the transformed electrocompetent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises at least 200 μg/mL of carbenicillin, wherein the antibiotic is present at a concentration at least twice the standard concentration of the antibiotic; and extracting plasmids from the second culture according to cGMP guidelines to produce a cGMP plasmid library.

In some aspects, a method of producing a cGMP plasmid library comprising a plurality of different plasmids comprises: transforming the plurality of different plasmids into a plurality of electrocompetent E. coli, wherein the plasmids comprise a nucleic acid encoding a carbenicillin resistance gene; contacting the transformed competent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises 200-400 μg/mL of carbenicillin, wherein the antibiotic is present at a concentration at least twice the standard concentration of the antibiotic; and extracting plasmids from the second culture according to cGMP guidelines to produce a cGMP plasmid library.

In some aspects, a method of producing a cGMP plasmid library comprising a plurality of different plasmids comprises: transforming the plurality of different plasmids into a plurality of electrocompetent E. coli, wherein the plasmids comprise a nucleic acid encoding a carbenicillin resistance gene; contacting the transformed electrocompetent E. coli with a first culture and achieving a minimum number of colonies per each of the different plasmids, wherein the first culture comprises carbenicillin; extracting the colonies from the first culture; growing the extracted colonies in a second culture according to cGMP guidelines, wherein the second culture comprises 200 μg/mL of carbenicillin; and extracting plasmids from the second culture according to cGMP guidelines to produce a cGMP plasmid library.

In some aspects, a method of producing a cGMP plasmid library comprising a plurality of different plasmids comprises: transforming the plurality of different plasmids into a plurality of electrocompetent E. coli, wherein the plasmids comprise a nucleic acid encoding a carbenicillin resistance gene; contacting the transformed electrocompetent E. coli with an agar plate and achieving at least 1000 colonies per each of the different plasmids, wherein the agar plate comprises carbenicillin; extracting the colonies from the agar plate; growing the extracted colonies in a liquid culture according to cGMP guidelines, wherein the liquid culture comprises about 200 μg/mL of carbenicillin; and extracting plasmids from the liquid culture according to cGMP guidelines to produce a cGMP plasmid library.

In some aspects, a method of producing a cGMP plasmid library comprising a plurality of different plasmids comprises: transforming the plurality of different plasmids into a plurality of electrocompetent E. coli, wherein the plasmids comprise a nucleic acid encoding a carbenicillin resistance gene; contacting the transformed electrocompetent E. coli with an agar plate and achieving at least 1000 colonies per each of the different plasmids, wherein the agar plate comprises carbenicillin; extracting the colonies from the agar plate; growing the extracted colonies in a liquid culture according to cGMP guidelines, wherein the liquid culture comprises 200 μg/mL of carbenicillin; and extracting plasmids from the liquid culture according to cGMP guidelines to produce a cGMP plasmid library.

### Plasmids and Plasmid Libraries

As used herein, a “plasmid library” refers to a plurality of different (e.g., encoding different sequences) plasmids. A “Current Good Manufacturing Practice (cGMP) plasmid library” refers to a plasmid library prepared according to cGMP guidelines (e.g., as described herein). In some embodiments, a “plasmid” as described herein refers to a circular piece of DNA comprising sequence elements for replication and expression of a polynucleotide (e.g., a guide RNA). In some embodiments, the plasmid comprises a nucleic acid encoding an antibiotic resistance gene.

In some embodiments, a plasmid library comprises at least about 5 (e.g., at least about 6, at least about 7, at least about 8, at least about 9, at least about 10, at least about 11, at least about 12, at least about 13, at least about 14, at least about 15, at least about 16, at least about 17, at least about 18, at least about 19 at least about 20, at least about 21, at least about 22, at least about 23, at least about 24, at least about 25, at least about 26, at least about 27, at least about 28, at least about 29, at least about 30, at least about 35, at least about 40, at least about 45, etc.) different plasmids. In some embodiments, a plasmid library comprises at least about 50 (e.g., at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 85, at least about 90, at least about 95, etc.) different plasmids. In some embodiments, the plasmid library comprises at least about 100 (e.g., at least about 110, at least about 120, at least about 130, at least about 140, at least about 150, at least about 160, at least about 170, at least about 180, at least about 190, at least about 200, at least about 210, at least about 220, at least about 230, at least about 240, etc.) different plasmids. In some embodiments, a plasmid library comprises at least about 500 (e.g., at least about 550, at least about 600, at least about 650, at least about 700, at least about 750, at least about 800, at least about 850, at least about 900, at least about 950, etc.) different plasmids. In some embodiments, a plasmid library comprises at least about 1000 (e.g., at least about 1100, at least about 1200, at least about 1300, at least about 1400, at least about 1500, at least about 1600, at least about 1700, at least about 1800, at least about 1900, at least about 2000, at least about 2500, etc.) different plasmids.

In some embodiments, a plasmid library comprises at least 5 (e.g., at least 6, at least 7, at least 8, at least 9, at least 10, at least 11, at least 12, at least 13, at least 14, at least 15, at least 16, at least 17, at least 18, at least 19 at least 20, at least 21, at least 22, at least 23, at least 24, at least 25, at least 26, at least 27, at least 28, at least 29, at least 30, at least 35, at least 40, at least 45, etc.) different plasmids. In some embodiments, a plasmid library comprises at least 50 (e.g., at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 85, at least 90, at least 95, etc.) different plasmids. In some embodiments, the plasmid library comprises at least 100 (e.g., at least 110, at least 120, at least 130, at least 140, at least 150, at least 160, at least 170, at least 180, at least 190, at least 200, at least 210, at least 220, at least 230, at least 240, etc.) different plasmids. In some embodiments, a plasmid library comprises at least 500 (e.g., at least 550, at least 600, at least 650, at least 700, at least 750, at least 800, at least 850, at least 900, at least 950, etc.) different plasmids. In some embodiments, a plasmid library comprises at least 1000 (e.g., at least 1100, at least 1200, at least 1300, at least 1400, at least 1500, at least 1600, at least 1700, at least 1800, at least 1900, at least 2000, at least 2500, etc.) different plasmids.

In some embodiments, a plasmid library comprises a plasmid that encodes an RNA or a protein. The term “protein” refers to a polymer of amino acids. In some embodiments, a plasmid library comprises a plasmid that encodes a therapeutic RNA or a therapeutic protein. A “therapeutic RNA” or “therapeutic protein” refers to an RNA or a protein that may be used to treat, ameliorate, inhibit, slow down, or stop the progression of a disease or disorder (e.g., cancer, a plasma cell disorder, or an autoimmune disorder). In some embodiments, a therapeutic RNA or therapeutic protein ameliorates symptoms or side effects of a disease or disorder.

In some embodiments, a plasmid library comprises a plasmid that encodes a variant of a gene that results in increased expression of the protein encoded by the gene. In some embodiments, a plasmid library comprises a plasmid that encodes a variant of a gene that results in decreased expression of the protein encoded by the gene.

Guide RNAs (gRNAs)

The present disclosure provides, in some aspects, methods of providing a Current Good Manufacturing Practice (cGMP) plasmid library comprising a plurality of different plasmids. For example, the plasmid library may comprise a plurality of different plasmids, each encoding a different guide RNA (gRNA). As used herein, the term “guide RNA (gRNA)” or “guide RNA (gRNA) polynucleotide” refers to a RNA polynucleotide or a DNA that encodes a guide RNA (gRNA). A guide RNA polynucleotide comprises a sequence that binds to a clustered regularly interspaced short palindromic repeats (CRISPR) protein or CRISPR-related protein and a sequence and comprises an additional sequence that is complementary to a target polynucleotide (i.e., a homology region). As used herein the term “CRISPR” may refer to a gene editing system that comprises a guide RNA component (e.g., a guide RNA encoded by a plasmid provided herein) and a CRISPR associated (Cas) protein component. The guide RNA polynucleotide may comprise a homology region that is complementary to a target gene and a stem loop region that is capable of binding to a Cas protein. The Cas protein may comprise a guide RNA binding site and nuclease activity. Methods for designing guide RNAs (e.g., selecting homology region sequences for targeting a specific gene) are also well known in the art as described in Liu, Guanqing L. et al., Computational and Structural Biotechnology Journal 18 (2020): 35-44, which is incorporated by reference in its entirety. In some embodiments, gRNAs (encoded by gRNA polynucleotides) are designed using CRISPick (portals.broadinstitute.org/gppx/crispick/public), which performs as described in Doench et al., Nature Biotechnology, 34 (2), 184-191 (2016) and Sanson et al., Nature Communications, 9 (1), 5416 (2018), both of which are incorporated by reference in their entirety.

### Bacteria and Transformation Thereof

Provided herein are methods of producing a Current Good Manufacturing Practice (cGMP) library comprising a plurality of different plasmids, the methods comprising transforming the plurality of different plasmids into a plurality of competent Escherichia coli (E. coli). Transforming bacteria involves the introduction of heterologous DNA into bacterial cells, such as linear DNA or plasmids as described herein.

Escherichia coli (E. coli) are the most ubiquitously used bacteria for transformation due to its fast growth rate, well-established culture methods, versatility, and the safety of laboratory strains. E. coli can be grown on multiple kinds of culture media, for example on agar plates or in liquid cultures, such as Luria-Bertani (LB) agar plates or broth, Terrific Buffer (TB) agar plates or broth, Super Optimal Broth with Catabolite repression (SOC) liquid medium (e.g., for recovery of E. coli after heat shock), on MacConkey agar plates, on X-Gal/IPTG Agar Plates, or using other solid or liquid substrates. In some embodiments, the methods provided herein comprise contacting transformed competent E. coli with a first culture and achieving a minimum number of colonies per each different plasmid in the plurality of different plasmids. In some embodiments, achieving a minimum number of colonies per each different plasmid provides a representation of each of the different plasmids in the plurality of plasmids when the plasmids are extracted to produce a cGMP plasmid library. In some embodiments, a minimum number of colonies is about a minimum number of colonies.

In some embodiments, a minimum number of colonies is about 100 colonies per each of the different plasmids. In some embodiments, a minimum number of colonies is about 200 colonies per each of the different plasmids. In some embodiments, a minimum number of colonies is about 300 colonies per each of the different plasmids. In some embodiments, a minimum number of colonies is about 400 colonies per each of the different plasmids. In some embodiments, a minimum number of colonies is about 500 colonies per each of the different plasmids. In some embodiments, a minimum number of colonies is about 600 colonies per each of the different plasmids. In some embodiments, a minimum number of colonies is about 700 colonies per each of the different plasmids. In some embodiments, a minimum number of colonies is about 800 colonies per each of the different plasmids. In some embodiments, a minimum number of colonies is about 900 colonies per each of the different plasmids. In some embodiments, a minimum number of colonies is about 1000 colonies per each of the different plasmids. In some embodiments, a minimum number of colonies is about 1100 colonies per each of the different plasmids. In some embodiments, a minimum number of colonies is about 1200 colonies per each of the different plasmids. In some embodiments, a minimum number of colonies is about 1300 colonies per each of the different plasmids. In some embodiments, a minimum number of colonies is about 1400 colonies per each of the different plasmids. In some embodiments, a minimum number of colonies is about 1500 colonies per each of the different plasmids. In some embodiments, a minimum number of colonies is about 2000 colonies per each of the different plasmids.

In some embodiments, a minimum number of colonies is 100 colonies per each of the different plasmids. In some embodiments, a minimum number of colonies is 200 colonies per each of the different plasmids. In some embodiments, a minimum number of colonies is 300 colonies per each of the different plasmids. In some embodiments, a minimum number of colonies is 400 colonies per each of the different plasmids. In some embodiments, a minimum number of colonies is 500 colonies per each of the different plasmids. In some embodiments, a minimum number of colonies is 600 colonies per each of the different plasmids. In some embodiments, a minimum number of colonies is 700 colonies per each of the different plasmids.

In some embodiments, a minimum number of colonies is 800 colonies per each of the different plasmids. In some embodiments, a minimum number of colonies is 900 colonies per each of the different plasmids. In some embodiments, a minimum number of colonies is 1000 colonies per each of the different plasmids. In some embodiments, a minimum number of colonies is 1100 colonies per each of the different plasmids. In some embodiments, a minimum number of colonies is 1200 colonies per each of the different plasmids. In some embodiments, a minimum number of colonies is 1300 colonies per each of the different plasmids. In some embodiments, a minimum number of colonies is 1400 colonies per each of the different plasmids. In some embodiments, a minimum number of colonies is 1500 colonies per each of the different plasmids. In some embodiments, a minimum number of colonies is 2000 colonies per each of the different plasmids.

In some embodiments, transforming comprises transforming competent E. coli. Competent E. coli are treated to enhance the uptake of exogenous DNA compared to E. coli that are not competent. Examples of competent types of E. coli include chemically-competent E. coli, which are transformed using, e.g., heat shock, and electrocompetent E. coli, which are transformed using, e.g., electroporation. Chemically-competent E. coli cells are commercially available and additionally can also be prepared, for example, by treating E. coli with a series of salt washes to disrupt the plasma membrane. Electrocompetent E. coli are also commercially available and additionally can be prepared, for example, by washing cells with cold deionized water and glycerol. In some embodiments, the competent E. coli comprise electrocompetent E. coli. In some embodiments, the competent E. coli comprise chemically-competent E. coli. In some embodiments, the competent E. coli comprise DH10B, TOP10, GB10BTM, GB5-alpha™, BL21, TG1, RR1, JM109, HB101, DH5a-E, DH12S, or Stb14™ competent E. coli cells. In some embodiments, the competent E. coli comprise Stb14™ competent E. coli cells.

Other species of bacteria (e.g., Bacillus subtilis) may be used in the methods provided herein

### Antibiotics and Antibiotic Resistance

The inclusion of antibiotic resistance genes in plasmids to be transformed into bacterial cells allows for the selection of cells that have successfully taken up a plasmid. An “antibiotic resistance gene” is a gene that protects a cell (e.g., a bacterial cell) from the cytotoxic effects of the corresponding antibiotic. For example, including a gene that confers resistance to carbenicillin in a plasmid allows for the selection of E. coli cells that have taken up the plasmid, by incorporating carbenicillin into culture medium (e.g., an agar plate or a liquid culture) on which the E. coli cells are grown.

In some embodiments, the antibiotic is any antibiotic effective in killing E. coli cells. These include, but are not limited to, antibiotics belonging to antibiotic classes such as beta-lactams, fluoroquinolones, aminoglycosides, sulfonamides and trimethoprim, tetracyclines, and macrolides. In some embodiments, the antibiotic is a beta-lactam antibiotic. In some embodiments, the beta-lactam antibiotic is a penicillin antibiotic. In some embodiments, the penicillin antibiotic is a carboxypenicillin antibiotic. In some embodiments, the carboxypenicillin antibiotic is carbenicillin.

A “standard concentration” of an antibiotic is a concentration at which an antibiotic is used in cell culture to prevent the growth of susceptible bacteria (e.g., a working concentration). Standard concentrations of antibiotics typically fall within the range of about 1 μg/mL to about 1000 μg/mL. Standard concentrations of antibiotics for use in cell culture are known in the art and can therefore be readily recognized by a person or ordinary skill. Exemplary antibiotics and their standard concentrations are provided in Table 1.

In some embodiments, the antibiotic is carbenicillin. In some embodiments, the antibiotic is ampicillin. In some embodiments, the antibiotic is bleomycin. In some embodiments, the antibiotic is chloramphenicol. In some embodiments, the antibiotic is D-cycloserine. In some embodiments, the antibiotic is erythromycin. In some embodiments, the antibiotic is gentamicin.

In some embodiments, the antibiotic is G418. In some embodiments, the antibiotic is kanamycin. In some embodiments, the antibiotic is nalidixic acid. In some embodiments, the antibiotic is novobiocin. In some embodiments, the antibiotic is rifampicin. In some embodiments, the antibiotic is spectinomycin. In some embodiments, the antibiotic is streptomycin. In some embodiments, the antibiotic is tetracycline. In some embodiments, the antibiotic is trimethoprim. In some embodiments, the antibiotic is zeocin.

In some aspects, provided herein are methods of producing a current Good Manufacturing Practice (cGMP) plasmid library comprising a plurality of different plasmids wherein the method comprises in part growing transformed bacterial cells in a liquid culture that comprises an antibiotic present at a concentration at least about twice (or at least 2x) the standard concentration of the antibiotic. In some embodiments, the antibiotic is present at a concentration at least about 1.5× the standard concentration. In some embodiments, the antibiotic is present at a concentration at least about 1.6× the standard concentration. In some embodiments, the antibiotic is present at a concentration at least about 1.7× the standard concentration. In some embodiments, the antibiotic is present at a concentration at least about 1.8× the standard concentration. In some embodiments, the antibiotic is present at a concentration at least about 1.9× the standard concentration. In some embodiments, the antibiotic is present at a concentration at least about 2.1× the standard concentration. In some embodiments, the antibiotic is present at a concentration at least about 2.2× the standard concentration. In some embodiments, the antibiotic is present at a concentration at least about 2.3× the standard concentration. In some embodiments, the antibiotic is present at a concentration at least about 2.4× the standard concentration. In some embodiments, the antibiotic is present at a concentration at least about 2.5× the standard concentration. In some embodiments, the antibiotic is present at a concentration at least about 2.6× the standard concentration. In some embodiments, the antibiotic is present at a concentration at least about 2.7× the standard concentration. In some embodiments, the antibiotic is present at a concentration at least about 2.8× the standard concentration. In some embodiments, the antibiotic is present at a concentration at least about 2.9× the standard concentration. In some embodiments, the antibiotic is present at a concentration at least about 3× the standard concentration. In some embodiments, the antibiotic is present at a concentration at least about 3.1× the standard concentration. In some embodiments, the antibiotic is present at a concentration at least about 3.2× the standard concentration. In some embodiments, the antibiotic is present at a concentration at least about 3.3× the standard concentration. In some embodiments, the antibiotic is present at a concentration at least about 3.4× the standard concentration. In some embodiments, the antibiotic is present at a concentration at least about 3.5× the standard concentration. In some embodiments, the antibiotic is present at a concentration at least about 3.6× the standard concentration. In some embodiments, the antibiotic is present at a concentration at least about 3.7× the standard concentration. In some embodiments, the antibiotic is present at a concentration at least about 3.8× the standard concentration. In some embodiments, the antibiotic is present at a concentration at least about 3.9× the standard concentration. In some embodiments, the antibiotic is present at a concentration at least about 4× the standard concentration. In some embodiments, the antibiotic is present at a concentration at least about 4.5× the standard concentration. In some embodiments, the antibiotic is present at a concentration at least about 5× the standard concentration. In some embodiments, the antibiotic is present at a concentration at least about 5.5× the standard concentration. In some embodiments, the antibiotic is present at a concentration at least about 6× the standard concentration. In some embodiments, the antibiotic is present at a concentration at least about 6.5× the standard concentration. In some embodiments, the antibiotic is present at a concentration at least about 7× the standard concentration. In some embodiments, the antibiotic is present at a concentration at least about 7.5× the standard concentration. In some embodiments, the antibiotic is present at a concentration at least about 8× the standard concentration. In some embodiments, the antibiotic is present at a concentration at least about 8.5× the standard concentration. In some embodiments, the antibiotic is present at a concentration at least about 9× the standard concentration. In some embodiments, the antibiotic is present at a concentration at least about 9.5× the standard concentration. In some embodiments, the antibiotic is present at a concentration at least about 10× the standard concentration.

In some embodiments, the antibiotic is present at a concentration at least 1.5× the standard concentration. In some embodiments, the antibiotic is present at a concentration at least 1.6× the standard concentration. In some embodiments, the antibiotic is present at a concentration at least 1.7× the standard concentration. In some embodiments, the antibiotic is present at a concentration at least 1.8× the standard concentration. In some embodiments, the antibiotic is present at a concentration at least 1.9× the standard concentration. In some embodiments, the antibiotic is present at a concentration at least 2.1× the standard concentration. In some embodiments, the antibiotic is present at a concentration at least 2.2× the standard concentration. In some embodiments, the antibiotic is present at a concentration at least 2.3× the standard concentration. In some embodiments, the antibiotic is present at a concentration at least 2.4× the standard concentration. In some embodiments, the antibiotic is present at a concentration at least 2.5× the standard concentration. In some embodiments, the antibiotic is present at a concentration at least 2.6× the standard concentration. In some embodiments, the antibiotic is present at a concentration at least 2.7× the standard concentration. In some embodiments, the antibiotic is present at a concentration at least 2.8× the standard concentration. In some embodiments, the antibiotic is present at a concentration at least 2.9× the standard concentration. In some embodiments, the antibiotic is present at a concentration at least 3× the standard concentration. In some embodiments, the antibiotic is present at a concentration at least 3.1× the standard concentration. In some embodiments, the antibiotic is present at a concentration at least 3.2× the standard concentration. In some embodiments, the antibiotic is present at a concentration at least 3.3× the standard concentration. In some embodiments, the antibiotic is present at a concentration at least 3.4× the standard concentration. In some embodiments, the antibiotic is present at a concentration at least 3.5× the standard concentration.

In some embodiments, the antibiotic is present at a concentration at least 3.6× the standard concentration. In some embodiments, the antibiotic is present at a concentration at least 3.7× the standard concentration. In some embodiments, the antibiotic is present at a concentration at least 3.8× the standard concentration. In some embodiments, the antibiotic is present at a concentration at least 3.9× the standard concentration. In some embodiments, the antibiotic is present at a concentration at least 4× the standard concentration. In some embodiments, the antibiotic is present at a concentration at least 4.5× the standard concentration. In some embodiments, the antibiotic is present at a concentration at least 5× the standard concentration. In some embodiments, the antibiotic is present at a concentration at least 5.5× the standard concentration. In some embodiments, the antibiotic is present at a concentration at least 6× the standard concentration. In some embodiments, the antibiotic is present at a concentration at least 6.5× the standard concentration. In some embodiments, the antibiotic is present at a concentration at least 7× the standard concentration. In some embodiments, the antibiotic is present at a concentration at least 7.5× the standard concentration. In some embodiments, the antibiotic is present at a concentration at least 8× the standard concentration. In some embodiments, the antibiotic is present at a concentration at least 8.5× the standard concentration. In some embodiments, the antibiotic is present at a concentration at least 9× the standard concentration. In some embodiments, the antibiotic is present at a concentration at least 9.5× the standard concentration. In some embodiments, the antibiotic is present at a concentration at least 10× the standard concentration.

In some embodiments, the antibiotic is present at a concentration at least about 50% greater than the standard concentration. In some embodiments, the antibiotic is present at a concentration at least about 60% greater than the standard concentration. In some embodiments, the antibiotic is present at a concentration at least about 70% greater than the standard concentration. In some embodiments, the antibiotic is present at a concentration at least about 80% greater than the standard concentration. In some embodiments, the antibiotic is present at a concentration at least about 90% greater than the standard concentration. In some embodiments, the antibiotic is present at a concentration at least about 100% greater than the standard concentration. In some embodiments, the antibiotic is present at a concentration at least about 110% greater than the standard concentration. In some embodiments, the antibiotic is present at a concentration at least about 120% greater than the standard concentration. In some embodiments, the antibiotic is present at a concentration at least about 130% greater than the standard concentration. In some embodiments, the antibiotic is present at a concentration at least about 140% greater than the standard concentration. In some embodiments, the antibiotic is present at a concentration at least about 150% greater than the standard concentration. In some embodiments, the antibiotic is present at a concentration at least about 160% greater than the standard concentration. In some embodiments, the antibiotic is present at a concentration at least about 170% greater than the standard concentration. In some embodiments, the antibiotic is present at a concentration at least about 180% greater than the standard concentration. In some embodiments, the antibiotic is present at a concentration at least about 190% greater than the standard concentration. In some embodiments, the antibiotic is present at a concentration at least about 200% greater than the standard concentration. In some embodiments, the antibiotic is present at a concentration at least about 210% greater than the standard concentration. In some embodiments, the antibiotic is present at a concentration at least about 220% greater than the standard concentration. In some embodiments, the antibiotic is present at a concentration at least about 230% greater than the standard concentration. In some embodiments, the antibiotic is present at a concentration at least about 240% greater than the standard concentration. In some embodiments, the antibiotic is present at a concentration at least about 250% greater than the standard concentration. In some embodiments, the antibiotic is present at a concentration at least about 260% greater than the standard concentration. In some embodiments, the antibiotic is present at a concentration at least about 270% greater than the standard concentration. In some embodiments, the antibiotic is present at a concentration at least about 280% greater than the standard concentration. In some embodiments, the antibiotic is present at a concentration at least about 290% greater than the standard concentration. In some embodiments, the antibiotic is present at a concentration at least about 300% greater than the standard concentration. In some embodiments, the antibiotic is present at a concentration at least about 350% greater than the standard concentration. In some embodiments, the antibiotic is present at a concentration at least about 400% greater than the standard concentration. In some embodiments, the antibiotic is present at a concentration at least about 450% greater than the standard concentration. In some embodiments, the antibiotic is present at a concentration at least about 500% greater than the standard concentration. In some embodiments, the antibiotic is present at a concentration at least about 550% greater than the standard concentration. In some embodiments, the antibiotic is present at a concentration at least about 600% greater than the standard concentration. In some embodiments, the antibiotic is present at a concentration at least about 650% greater than the standard concentration. In some embodiments, the antibiotic is present at a concentration at least about 700% greater than the standard concentration. In some embodiments, the antibiotic is present at a concentration at least about 750% greater than the standard concentration. In some embodiments, the antibiotic is present at a concentration at least about 800% greater than the standard concentration. In some embodiments, the antibiotic is present at a concentration at least about 850% greater than the standard concentration. In some embodiments, the antibiotic is present at a concentration at least about 900% greater than the standard concentration. In some embodiments, the antibiotic is present at a concentration at least about 950% greater than the standard concentration. In some embodiments, the antibiotic is present at a concentration at least about 1000% greater than the standard concentration.

In some embodiments, the antibiotic is present at a concentration at least 50% greater than the standard concentration. In some embodiments, the antibiotic is present at a concentration at least 60% greater than the standard concentration. In some embodiments, the antibiotic is present at a concentration at least 70% greater than the standard concentration. In some embodiments, the antibiotic is present at a concentration at least 80% greater than the standard concentration. In some embodiments, the antibiotic is present at a concentration at least 90% greater than the standard concentration. In some embodiments, the antibiotic is present at a concentration at least 100% greater than the standard concentration. In some embodiments, the antibiotic is present at a concentration at least 110% greater than the standard concentration. In some embodiments, the antibiotic is present at a concentration at least 120% greater than the standard concentration. In some embodiments, the antibiotic is present at a concentration at least 130% greater than the standard concentration. In some embodiments, the antibiotic is present at a concentration at least 140% greater than the standard concentration. In some embodiments, the antibiotic is present at a concentration at least 150% greater than the standard concentration. In some embodiments, the antibiotic is present at a concentration at least 160% greater than the standard concentration. In some embodiments, the antibiotic is present at a concentration at least 170% greater than the standard concentration. In some embodiments, the antibiotic is present at a concentration at least 180% greater than the standard concentration. In some embodiments, the antibiotic is present at a concentration at least 190% greater than the standard concentration. In some embodiments, the antibiotic is present at a concentration at least 200% greater than the standard concentration. In some embodiments, the antibiotic is present at a concentration at least 210% greater than the standard concentration. In some embodiments, the antibiotic is present at a concentration at least 220% greater than the standard concentration. In some embodiments, the antibiotic is present at a concentration at least 230% greater than the standard concentration. In some embodiments, the antibiotic is present at a concentration at least 240% greater than the standard concentration. In some embodiments, the antibiotic is present at a concentration at least 250% greater than the standard concentration. In some embodiments, the antibiotic is present at a concentration at least 260% greater than the standard concentration. In some embodiments, the antibiotic is present at a concentration at least 270% greater than the standard concentration. In some embodiments, the antibiotic is present at a concentration at least 280% greater than the standard concentration. In some embodiments, the antibiotic is present at a concentration at least 290% greater than the standard concentration. In some embodiments, the antibiotic is present at a concentration at least 300% greater than the standard concentration. In some embodiments, the antibiotic is present at a concentration at least 350% greater than the standard concentration. In some embodiments, the antibiotic is present at a concentration at least 400% greater than the standard concentration. In some embodiments, the antibiotic is present at a concentration at least 450% greater than the standard concentration. In some embodiments, the antibiotic is present at a concentration at least 500% greater than the standard concentration. In some embodiments, the antibiotic is present at a concentration at least 550% greater than the standard concentration. In some embodiments, the antibiotic is present at a concentration at least 600% greater than the standard concentration. In some embodiments, the antibiotic is present at a concentration at least 650% greater than the standard concentration. In some embodiments, the antibiotic is present at a concentration at least 700% greater than the standard concentration. In some embodiments, the antibiotic is present at a concentration at least 750% greater than the standard concentration. In some embodiments, the antibiotic is present at a concentration at least 800% greater than the standard concentration. In some embodiments, the antibiotic is present at a concentration at least 850% greater than the standard concentration. In some embodiments, the antibiotic is present at a concentration at least 900% greater than the standard concentration. In some embodiments, the antibiotic is present at a concentration at least 950% greater than the standard concentration. In some embodiments, the antibiotic is present at a concentration at least 1000% greater than the standard concentration.

In some embodiments, an agar plate or liquid culture comprises an antibiotic present at a concentration at least twice the standard concentration of the antibiotic. In some embodiments, the antibiotic and corresponding concentration is: ampicillin: at least about 200 μg/mL; carbenicillin: at least about 200 μg/mL; kanamycin: at least about 200 μg/mL; spectinomycin: at least about 100 μg/mL; streptomycin: at least about 200 μg/mL; bleomycin: at least about 200 μg/mL; erythromycin: at least about 200 μg/mL; chloramphenicol: at least about 50 μg/mL;

tetracycline: at least about 20 μg/mL; zeocin: at least about 200 g/mL; D-cycloserine: at least about 400 μg/mL; gentamicin: at least about 30 μg/mL; G418: at least about 800 μg/mL;

nalidixic acid: at least about 60 g/mL; rifampicin: at least about 100 μg/mL; or trimethoprim: at least about 400 μg/mL. In some embodiments, the antibiotic and corresponding concentration is ampicillin: at least about 200 μg/mL. In some embodiments, the antibiotic and corresponding concentration is carbenicillin: at least about 200 μg/mL. In some embodiments, the antibiotic and corresponding concentration is kanamycin: at least about 200 μg/mL. In some embodiments, the antibiotic and corresponding concentration is spectinomycin: at least about 100 μg/mL. In some embodiments, the antibiotic and corresponding concentration is streptomycin: at least about 200 μg/mL. In some embodiments, the antibiotic and corresponding concentration is bleomycin: at least about 200 g/mL. In some embodiments, the antibiotic and corresponding concentration is erythromycin: at least about 200 μg/mL. In some embodiments, the antibiotic and corresponding concentration is chloramphenicol: at least about 50 μg/mL. In some embodiments, the antibiotic and corresponding concentration is tetracycline: at least about 20 μg/mL. In some embodiments, the antibiotic and corresponding concentration is zeocin: at least about 200 μg/mL. In some embodiments, the antibiotic and corresponding concentration is D-cycloserine: at least about 400 μg/mL. In some embodiments, the antibiotic and corresponding concentration is gentamicin: at least about 30 μg/mL. In some embodiments, the antibiotic and corresponding concentration is G418: at least about 800 g/mL. In some embodiments, the antibiotic and corresponding concentration is nalidixic acid: at least about 60 μg/mL. In some embodiments, the antibiotic and corresponding concentration is rifampicin: at least about 100 μg/mL. In some embodiments, the antibiotic and corresponding concentration is trimethoprim: at least about 400 μg/mL. In some embodiments, the antibiotic and corresponding concentration is trimethoprim: at least about 400 μg/mL. In some embodiments, the antibiotic is carbenicillin and the corresponding concentration is about 200-400 μg/mL. In some embodiments, the antibiotic is carbenicillin and the corresponding concentration is at least about 150 μg/mL. In some embodiments, the antibiotic is carbenicillin and the corresponding concentration is about 200 μg/mL.

In some embodiments, an agar plate or liquid culture comprises an antibiotic present at a concentration at least twice the standard concentration of the antibiotic. In some embodiments, the antibiotic and corresponding concentration is: ampicillin: at least 200 μg/mL; carbenicillin: at least 200 μg/mL; kanamycin: at least 200 μg/mL; spectinomycin: at least 100 μg/mL; streptomycin: at least 200 μg/mL; bleomycin: at least 200 μg/mL; erythromycin: at least 200 μg/mL; chloramphenicol: at least 50 μg/mL; tetracycline: at least 20 μg/mL; zeocin: at least 200 μg/mL; D-cycloserine: at least 400 μg/mL; gentamicin: at least 30 g/mL; G418: at least 800 μg/mL; nalidixic acid: at least 60 μg/mL; rifampicin: at least 100 μg/mL; or trimethoprim: at least 400 μg/mL. In some embodiments, the antibiotic and corresponding concentration is ampicillin: at least 200 μg/mL. In some embodiments, the antibiotic and corresponding concentration is carbenicillin: at least 200 g/mL. In some embodiments, the antibiotic and corresponding concentration is kanamycin: at least 200 μg/mL. In some embodiments, the antibiotic and corresponding concentration is spectinomycin: at least 100 μg/mL. In some embodiments, the antibiotic and corresponding concentration is streptomycin: at least 200 μg/mL. In some embodiments, the antibiotic and corresponding concentration is bleomycin: at least 200 μg/mL. In some embodiments, the antibiotic and corresponding concentration is erythromycin: at least 200 μg/mL. In some embodiments, the antibiotic and corresponding concentration is chloramphenicol: at least 50 μg/mL. In some embodiments, the antibiotic and corresponding concentration is tetracycline: at least 20 μg/mL. In some embodiments, the antibiotic and corresponding concentration is zeocin: at least 200 μg/mL. In some embodiments, the antibiotic and corresponding concentration is D-cycloserine: at least 400 g/mL. In some embodiments, the antibiotic and corresponding concentration is gentamicin: at least 30 g/mL. In some embodiments, the antibiotic and corresponding concentration is G418: at least 800 μg/mL. In some embodiments, the antibiotic and corresponding concentration is nalidixic acid: at least 60 μg/mL. In some embodiments, the antibiotic and corresponding concentration is rifampicin: at least 100 μg/mL. In some embodiments, the antibiotic and corresponding concentration is trimethoprim: at least 400 μg/mL. In some embodiments, the antibiotic and corresponding concentration is trimethoprim: at least 400 μg/mL. In some embodiments, the antibiotic is carbenicillin and the corresponding concentration is 200-400 μg/mL. In some embodiments, the antibiotic is carbenicillin and the corresponding concentration is at least 150 μg/mL. In some embodiments, the antibiotic is carbenicillin and the corresponding concentration is 200 μg/mL. Exemplary antibiotics and their corresponding concentrations are shown in Table 1.

TABLE 1
Exemplary antibiotics and concentrations for use.

| Antibiotic | Standard concentration | | Double concentration | |
|---|---|---|---|---|
| Ampicillin | 100 | μg/mL | 200 | μg/mL |
| Carbenicillin | 100 | μg/mL | 200 | μg/mL |
| Kanamycin | 100 | μg/mL | 200 | μg/mL |
| Spectinomycin | 50 | μg/mL | 100 | μg/mL |
| Streptomycin | 100 | μg/mL | 200 | μg/mL |
| Bleomycin | 100 | μg/mL | 200 | μg/mL |
| Erythromycin | 100 | μg/mL | 200 | μg/mL |
| Chloramphenicol | 25 | μg/mL | 50 | μg/mL |
| Tetracycline | 10 | μg/mL | 20 | μg/mL |
| Zeocin | 100 | μg/mL | 200 | μg/mL |
| D-cycloserine | 200 | μg/mL | 400 | μg/mL |
| Gentamicin | 15 | μg/mL | 30 | μg/mL |
| G418 | 400 | μg/mL | 800 | μg/mL |
| Nalidixic acid | 30 | μg/mL | 60 | μg/mL |
| Rifampicin | 50 | μg/mL | 100 | μg/mL |
| Trimethoprim | 200 | μg/mL | 400 | μg/mL |

### General Definitions

The terms “decrease,” “reduced,” “reduction,” or “inhibit” are all used herein to mean a decrease by a statistically significant amount. In some embodiments, “reduce,” “reduction,” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. “Complete inhibition” is a 100% inhibition as compared to a reference level. Where applicable, a decrease can be preferably down to a level accepted as within the range of normal for an individual without a given disorder. The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

In some embodiments, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. In some embodiments, an “engineered cell” refers to a cell with an added, deleted and/or altered gene (e.g. using a guide RNA produced by a method provided herein). In some embodiments, an “engineered nucleic acid”, “engineered nucleic acid molecule”, or “engineered polynucleotide” refers to a nucleic acid, nucleic acid molecule, or polynucleotide that contains one or more human-designed alterations.

The term “cell” or “engineered cell” and their grammatical equivalents as used herein can refer to a cell of human or non-human animal origin.

The term “polynucleotide” is used herein interchangeably with “nucleic acid molecule” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA,

RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. In some embodiments, the nucleic acid molecule is a heterologous nucleic acid molecule. As used herein the term, “heterologous nucleic acid molecule” refers to a nucleic acid molecule that does not naturally exist within a given cell.

The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” may refer to a vector that directs expression of an RNA or protein from sequences linked to transcriptional regulatory sequences on the vector. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. As used herein, the term “viral vector” may refer to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art. Other than in the operating examples, or where otherwise indicated, all numbers expressing quantities of ingredients or reaction conditions used herein should be understood as modified in all instances by the term “about.”

The terms “about” or “approximately” when used in connection with a value can mean that the value or a statement reciting the value encompasses a range within +1%, +2%, +3%, +4%, +5%, +6%, +7%, +8%, +9%, +10%, +1-5%, +2-7%, +3-8%, +4-9%, or +5-10% of the value.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology, as set forth herein.

### Examples

### Example 1. Large-Scale cGMP Plasmid Library Production

Compliance with current Good Manufacturing Practice (cGMP) guidelines ensure that clinical products are safe for use and have the components and efficacy they purport to have. However, standard cGMP guidelines for producing plasmids are limited to single plasmids and thus do not allow for large-scale transduction or transformation with plasmid libraries. Presented herein are novel processes for maintaining representation of an entire library while adhering to cGMP guidelines.

### Methods and Results

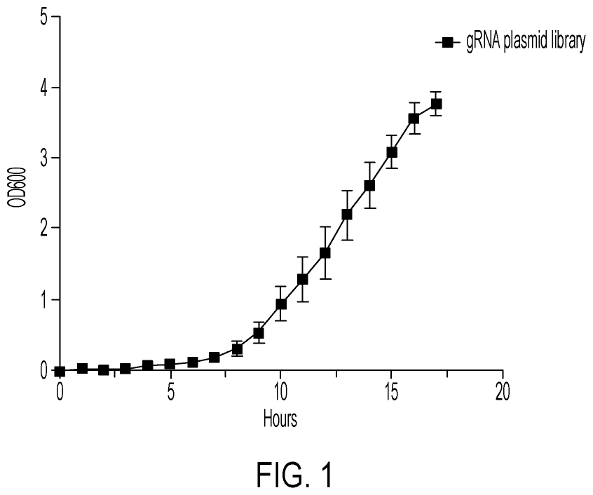

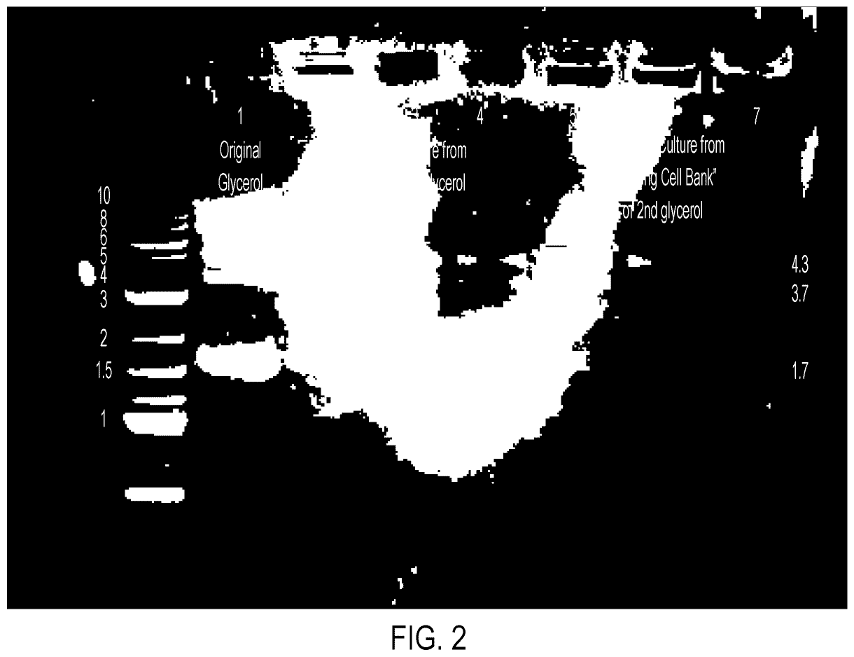

A plasmid library of 1,180 unique plasmids containing 1,180 unique Clustered Regularly Interspaced Short Palindromic Repeats (CRISPR) guide RNAs (gRNAs) and a carbenicillin-resistance gene was prepared. Initially, plasmids were transformed into chemically-competent E. coli, according to standard protocols that adhere to cGMP, and E. coli were grown in agar plates containing carbenicillin. Glycerol stocks were prepared from bacterial colonies isolated from the agar plates. Initial analysis of plasmid yield was performed by scraping glycerol stocks and growing each scrape in 15 mL of Terrific Buffer (TB) containing 100 μg/mL of carbenicillin grown at 30 °C. Analysis of the E. coli growth curve showed that, at 16 hours, the E. coli culture was just beginning to leave the log phase (as measured by optical density, OD) (FIG. 1), resulting in a very low plasmid yield (FIG. 2), ultimately unusable for large-scale transduction or transformation of immune effector cells.

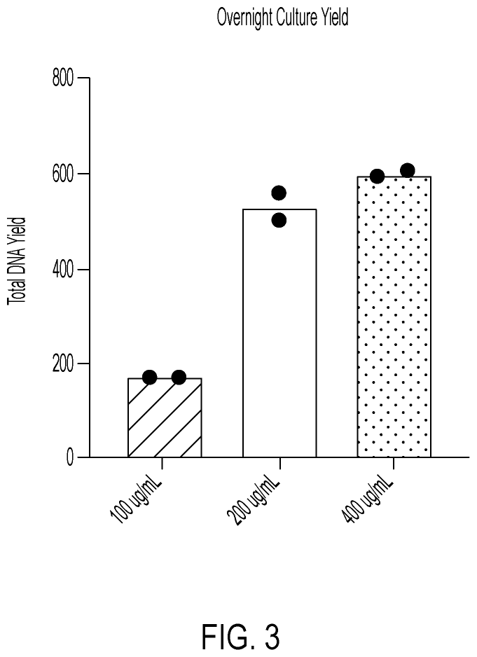

Next, several parameters were adjusted to improve plasmid yield. Plasmids were transformed into electrocompetent E. coli, which were plated on an agar plate containing carbenicillin. Agar plates were incubated to allow bacterial growth to reach an average colony cover of at least 1,000 colonies per unique plasmid, after which glycerol stock pools containing the colonies were prepared. Glycerol stocks were scraped and grown overnight in 250 mL of TB containing 100 μg/mL, 200 μg/mL, or 400 μg/mL of carbenicillin. Cultures containing carbenicillin concentrations of 200 μg/mL and 400 μg/mL both led to markedly improved DNA yield (FIG. 3).

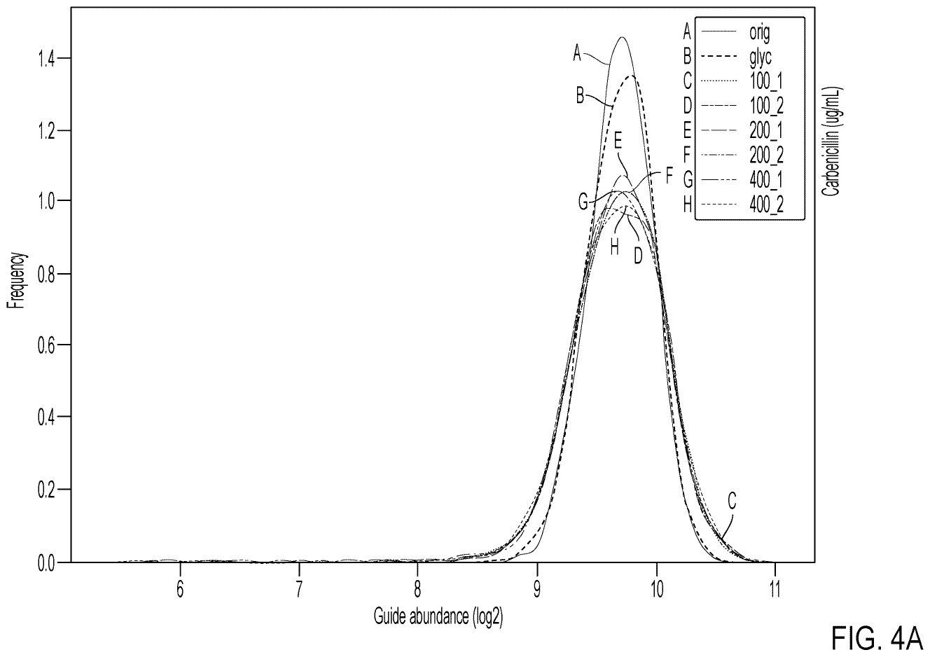

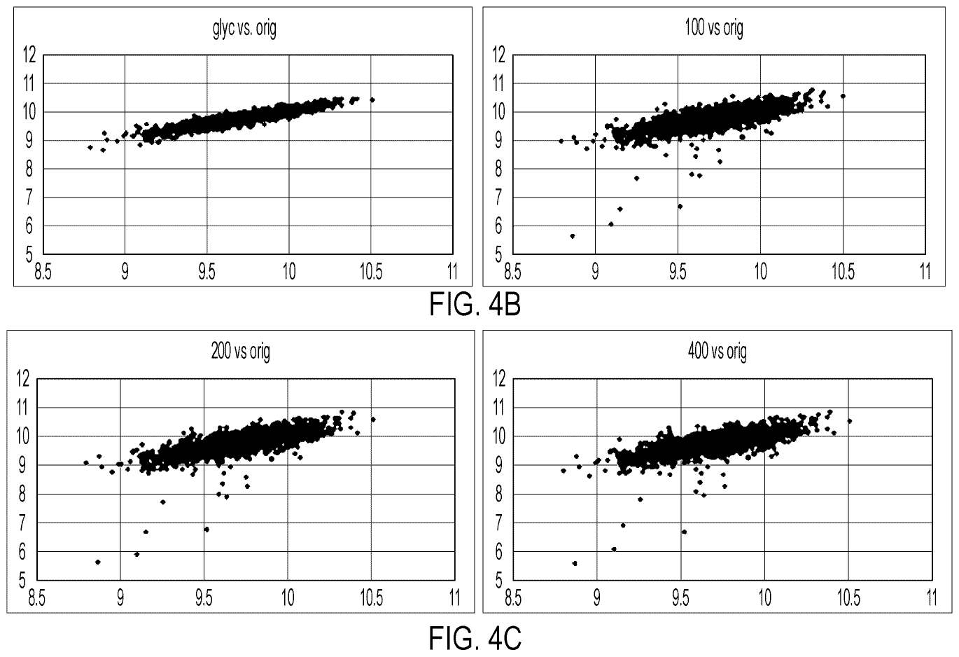

To determine whether DNA yield also correlated with full coverage of the gRNA library, plasmid preps were sequenced and results were compared to the original plasmid library. Plasmid preps were found to achieve similar guide abundance compared to the original library (FIGS. 4A-4C).

To determine whether above preparations would achieve similar results using cGMP guidelines, glycerol stocks were prepared and shipped to a contract research organization (CRO). Each glycerol scrape of approximately 20 μL was grown for 6-8 hours at 37 °C in 15 mL of Terrific Buffer (TB) containing 200 μg/mL of carbenicillin. Thereafter, the liquid culture was transferred to 12L of TB containing 200 μg/mL of carbenicillin, from which the plasmids were extracted according to cGMP guidelines.

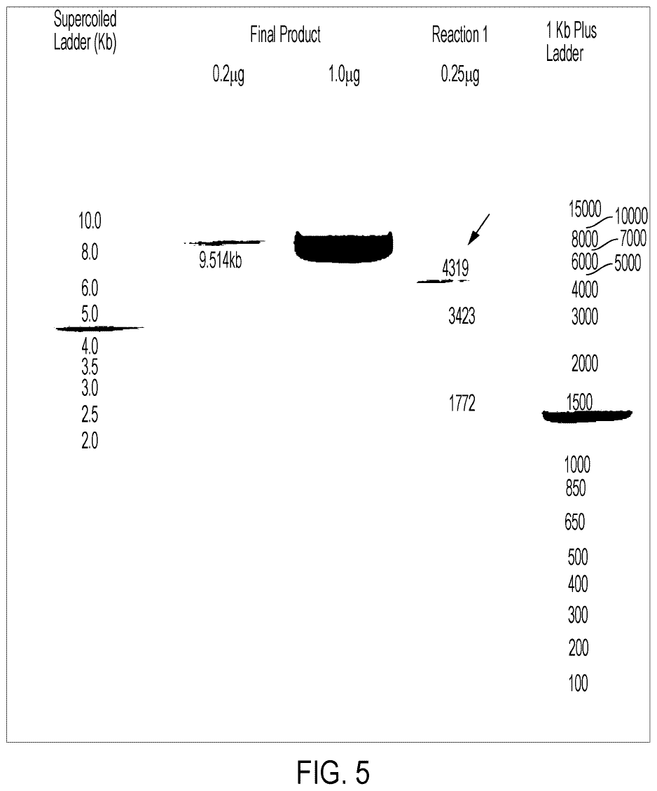

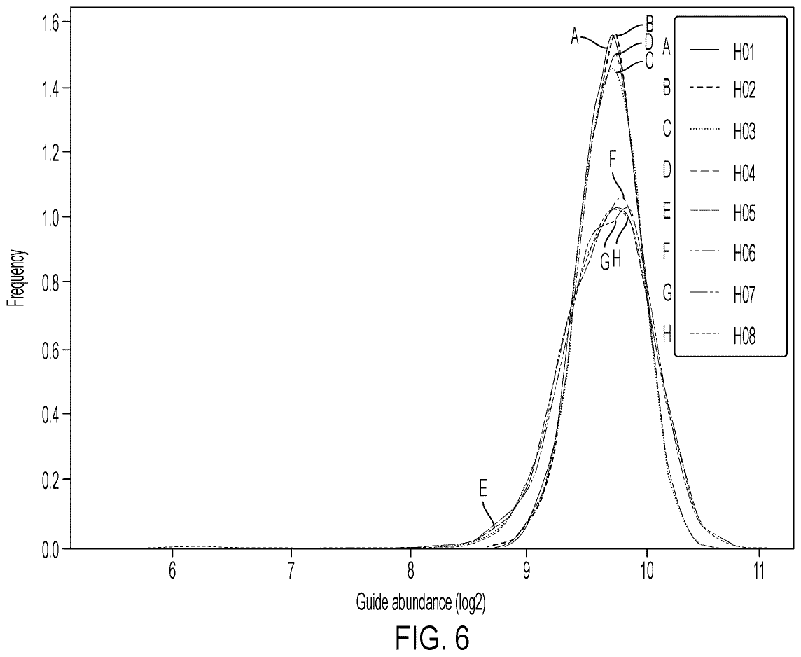

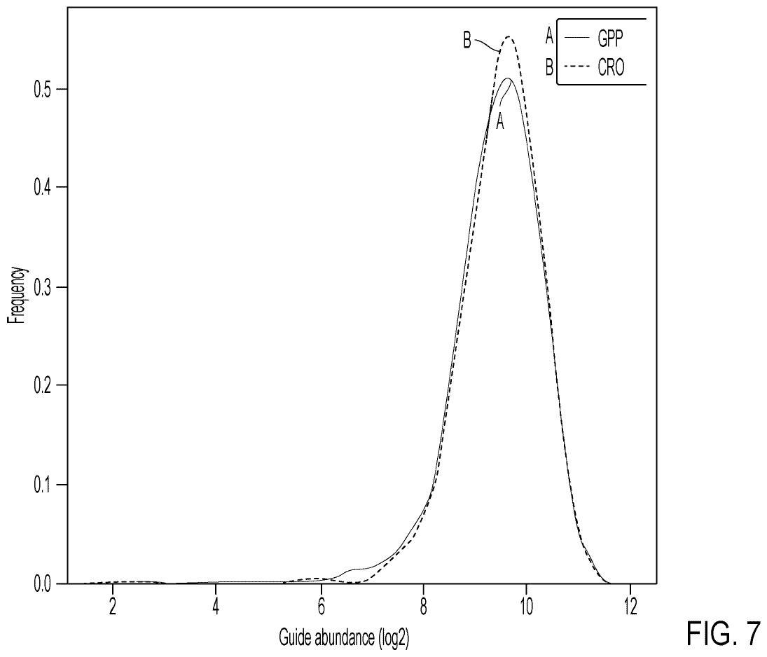

Treatment of pooled gRNA library plasmid preps with ScaI and ExoRI restriction enzymes resulted in DNA fragments of expected sizes (FIG. 5). Comparison of gRNA distribution as prepared according to cGMP guidelines showed similar distribution of guide abundance compared to plasmid pool preps prepared as above (FIG. 6). Critically, gRNA distributions in primary human T cells transduced with virus made from pooled plasmids prepared using the methods provided herein according to both cGMP guidelines and in a standard research laboratory (non-GMP) both resulted in similar guide abundance and frequency.

Taken together, the data demonstrate that the methods provided herein allow for large-scale plasmid library preparation according to cGMP guidelines, which permits large-scale transduction or transformation of immune effector cells (such as T cells) for use in humans, which was previously limited by standard protocols that adhere to cGMP guidelines, which are drawn to production of homogenous plasmids (e.g., plasmids encoding the same sequence). The methods could also be used to make large-scale preps of CRISPR screens or other library-based approaches for in vivo testing.

What is claimed is:

1. A method of producing a Current Good Manufacturing Practice (cGMP) plasmid library comprising a plurality of different plasmids, the method comprising:
transforming the plurality of different plasmids into a plurality of competent E. coli, wherein the plasmids comprise a nucleic acid encoding an antibiotic resistance gene;
contacting the transformed competent E. coli with a first culture and achieving a minimum number of colonies per each of the different plasmids, wherein the first culture comprises an antibiotic corresponding to the antibiotic resistance gene;
extracting the colonies from the first culture;
growing the extracted colonies in a second culture according to cGMP guidelines, wherein the second culture comprises an antibiotic that corresponds to the antibiotic resistance gene, wherein the antibiotic is present at a concentration at least twice the standard concentration of the antibiotic; and
extracting plasmids from the second culture according to cGMP guidelines to produce a cGMP plasmid library.

2. The method of claim 1, wherein the minimum number of colonies is about 100 colonies per each of the different plasmids.

3. The method of claim 1, wherein the minimum number of colonies is about 1000 colonies per each of the different plasmids.

4. The method of any one of claims 1-3, wherein the plasmid library comprises a plasmid that encodes a RNA or a protein.

5. The method of any one of claims 1-4, wherein the plasmid library comprises a plasmid that encodes a therapeutic RNA or a therapeutic protein.

6. The method of any one of claims 1-5, wherein the plasmid library comprises at least about 5 different plasmids.

7. The method of any one of claims 1-5, wherein the plasmid library comprises at least about 50 different plasmids.

8. The method of any one of claims 1-5, wherein the plasmid library comprises at least about 100 different plasmids.

9. The method of any one of claims 1-5, wherein the plasmid library comprises at least about 250 different plasmids.

10. The method of any one of claims 1-5, wherein the plasmid library comprises at least about 500 different plasmids.

11. The method of any one of claims 1-5, wherein the plasmid library comprises at least about 1000 different plasmids.

12. The method of any one of claims 1-11, wherein the competent E. coli comprise electrocompetent E. coli.

13. The method of any one of claims 1-11, wherein the competent E. coli comprise chemically-competent E. coli.

14. The method of any one of claims 1-13, wherein the antibiotic is any one of ampicillin, carbenicillin, kanamycin, specinomycin, streptomycin, bleomycin, erythromycin, chloramphenicol, tetracycline, zeocin, D-cycloserine, gentamicin, G418, nalidixic acid, rifampicin, trimethoprim.

15. The method of claim any one of claims 1-13, wherein the antibiotic is a beta-lactam antibiotic.

16. The method of claim 15, wherein the beta-lactam antibiotic is a penicillin antibiotic.

17. The method of claim 16, wherein the penicillin antibiotic is a carboxypenicillin antibiotic.

18. The method of claim 17, wherein the carboxypenicillin antibiotic is carbenicillin.

19. The method of claim 18, wherein the carbenicillin concentration is at least about 150 μg/mL.

20. The method of claim 19, wherein the carbenicillin concentration is at least about 200 μg/mL.

21. The method of any one of claims 1-14, wherein the antibiotic and corresponding concentration is ampicillin: at least about 200 μg/mL; carbenicillin: at least about 200 μg/mL;
kanamycin: at least about 200 g/mL; spectinomycin: at least about 100 g/mL; streptomycin: at least about 200 g/mL; bleomycin: at least about 200 μg/mL; erythromycin: at least about 200 μg/mL; chloramphenicol: at least about 50 μg/mL; tetracycline: at least about 20 μg/mL; zeocin:
at least about 200 μg/mL; D-cycloserine: at least about 400 μg/mL; gentamicin: at least about 30 μg/mL; G418: at least about 800 μg/mL; nalidixic acid: at least about 60 μg/mL; rifampicin: at least 100 about μg/mL; or trimethoprim: at least about 400 μg/mL.

22. A method of producing a Current Good Manufacturing Practice (cGMP) plasmid library comprising a plurality of different plasmids, the method comprising:
transforming the plurality of different plasmids into a plurality of electrocompetent E. coli, wherein the plasmids comprise a nucleic acid encoding a carbenicillin resistance gene;
contacting the transformed competent E. coli with a first culture and achieving a minimum number of colonies per each of the different plasmids, wherein the first culture comprises carbenicillin;
extracting the colonies from the first culture;
growing the extracted colonies in a second culture according to cGMP guidelines, wherein the second culture comprises at least about 200 μg/mL of carbenicillin; and
extracting plasmids from the second culture according to cGMP guidelines to produce a cGMP plasmid library.

23. A method of producing a Current Good Manufacturing Practice (cGMP) plasmid library comprising a plurality of different plasmids, the method comprising:
transforming the plurality of different plasmids into a plurality of electrocompetent E. coli, wherein the plasmids comprise a nucleic acid encoding a carbenicillin resistance gene;
contacting the transformed electrocompetent E. coli with a first culture and achieving a minimum number of colonies per each of the different plasmids, wherein the first culture comprises carbenicillin;
extracting the colonies from the first culture;
growing the extracted colonies in a second culture according to cGMP guidelines, wherein the second culture comprises about 200-400 μg/mL of carbenicillin; and
extracting plasmids from the second culture according to cGMP guidelines to produce a cGMP plasmid library.

24. A method of producing a Current Good Manufacturing Practice (cGMP) plasmid library comprising a plurality of different plasmids, the method comprising:
transforming the plurality of different plasmids into a plurality of electrocompetent E. coli, wherein the plasmids comprise a nucleic acid encoding a carbenicillin resistance gene;
contacting the transformed competent E. coli with a first culture and achieving a minimum number of colonies per each of the different plasmids, wherein the first culture comprises carbenicillin;
extracting the colonies from the first culture;
growing the extracted colonies in a second culture according to cGMP guidelines, wherein the second culture comprises about 200 μg/mL of carbenicillin; and
extracting plasmids from the second culture according to cGMP guidelines to produce a cGMP plasmid library.

25. A method of producing a Current Good Manufacturing Practice (cGMP) plasmid library comprising a plurality of different plasmids, the method comprising:
transforming the plurality of different plasmids into a plurality of electrocompetent E. coli, wherein the plasmids comprise a nucleic acid encoding a carbenicillin resistance gene;
contacting the transformed competent E. coli with an agar plate and achieving at least about 1000 colonies per each of the different plasmids, wherein the agar plate comprises carbenicillin;
extracting the colonies from the agar plate;
growing the extracted colonies in a liquid culture according to cGMP guidelines, wherein the liquid culture comprises about 200 μg/mL of carbenicillin; and
extracting plasmids from the liquid culture according to cGMP guidelines to produce a cGMP plasmid library.

26. The method of any one of claims 22-24, wherein the minimum number of colonies is about 100 colonies per each of the different plasmids.

27. The method of any one of claims 22-24, wherein the minimum number of colonies is about 1000 colonies per each of the different plasmids.
